(19) United States
(12) Patent Application Publication
(10) Pub. No.: US 2005/0135632 A1
(43) Pub. Date: Jun. 23, 2005

(54) Method and apparatus for detecting and locating noise sources not correlated

(75) Inventors: Alfred Permuy, Rueil Malmaison (FR); Joel Millet, Montalieu-Vercieu (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: METRAVIB R.D.S.

(21) Appl. No.: 10/736,895

(22) Filed: Dec. 17, 2003

(51) Int. Cl. A61F 11/06; G10K 11/16; H03B 29/00

(52) U.S. Cl. 381/71.2; 381/71.13

(57) ABSTRACT

According to the invention, the method of detecting and locating sources of noise each emitting respective signals Sⱼ with j=1 to M, detection being performed using sensors each delivering a respective time-varying electrical signal sᵢ with i varying from 1 to N, consists in taking the time-varying electrical signals delivered by the sensors, each signal sᵢ(t) delivered by a sensor being the sum of the signals Sⱼ emitted by the noise sources, in amplifying and filtering the time-varying electrical signals as taken, in digitizing the electrical signals, in calculating the functional

$$f ( {n}_{1} , … , {n}_{j} , … , {n}_{N} ) = {\sum }_{k \neq 1}^{} {R}_{k1}$$

with coefficients Rₖₗ being a function of the vectors nⱼ giving the directions of the noise sources, and in minimizing the functional f in such a manner as to determine the directions nⱼ of the noise sources.

### FIELD OF THE INVENTION

The present invention relates to detecting and locating sources of noise in the general sense, using sensors that are appropriate for the nature of the noise source.

The invention relates to a method of detecting and locating noise sources disposed in a space of one, two, or three dimensions and optionally correlated with one another, and presenting emission spectra of narrow or broad band.

The invention finds particularly advantageous applications in the field of locating sources of noise optionally accompanied by echo and coming, for example, from vehicles, ships, aircraft, or firearms.

### BACKGROUND OF THE INVENTION

In numerous applications, a need arises to be able to locate in relatively accurate manner a source of noise in order to take measures to neutralize it. Numerous solutions are known in the prior art for acoustically locating noise sources. The main known solutions make use of techniques for correlating signals delivered by detection sensors.

Those techniques present the drawback of being particularly sensitive to interfering noise occurring in the environment of the measurement sensors. Furthermore, it must be considered that those techniques constitute specific methods that are adapted to each application under consideration.

The technique in most widespread use involves antennas having a large number of sensors (several hundred) and a large computer system implementing beam forming so as to aim in a given direction in order to increase the signal-to-noise ratio. That method does not make any a priori assumption concerning the number of sources and any possible correlation between them, which leads to a loss of resolution.

### OBJECTS AND SUMMARY OF THE INVENTION

There therefore exists a need to have a general method of detecting and locating noise sources in space, when the number of noise sources is small and is known or overestimated.

The invention seeks to satisfy this need by proposing a method of detecting and locating noise sources by means of sensors adapted to the nature of the noise source, the method presenting low implementation costs.

To achieve this object, the method of the invention consists:
in taking the time-varying electrical signals delivered by the sensors (Yᵢ), each signal sᵢ(t) delivered by a sensor being the sum of the signals Sⱼ emitted by the noise sources (Xⱼ);
in amplifying and filtering the time-varying electrical signals as taken;
in digitizing the electrical signals;
in calculating the functional

$$f ( {n}_{1} , … , {n}_{j} , … , {n}_{N} ) = {\sum }_{k \neq 1}^{} {R}_{k1}$$

with the coefficients Rₖₗ being a function of the vectors nⱼ giving the directions of the noise sources; and
in minimizing the functional in such a manner as to determine the directions of the noise sources.

### BRIEF DESCRIPTION OF THE DRAWINGS

Various other characteristics appear from the description given below with reference to the accompanying drawing which shows embodiments and implementations of the invention as non-limiting examples.

FIG. 1 is a diagram showing the principle of the detection method of the invention.

FIG. 2 is a diagram showing a detail characteristic to the method of the invention.

FIG. 3 is a diagram showing the method of locating two noise sources using two sensors.

### MORE DETAILED DESCRIPTION

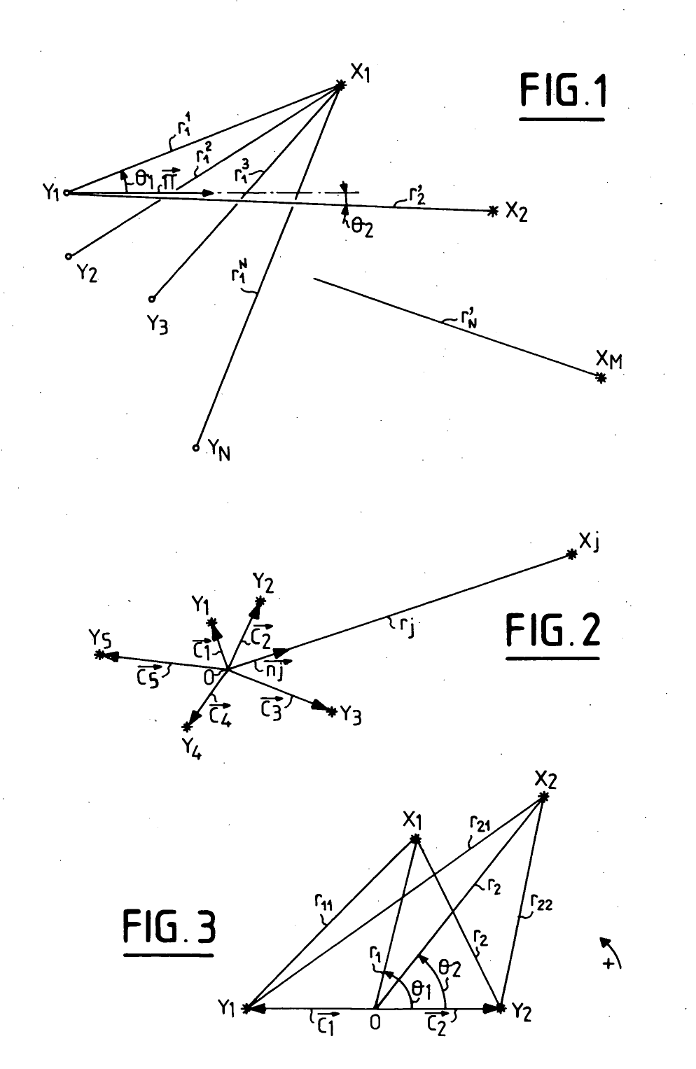

As can be seen in FIG. 1, the method of the invention consists in locating noise sources Xₗ, X₂, . . . , Xⱼ, . . . , X_{M} where j varies over the range 1 to M, the sources being distributed in space and each emitting a respective signal Sⱼ with j varying in the range 1 to M. The method of the invention consists in locating the noise sources Xⱼ using sound wave or vibration sensors Y₁, Y₂, . . . , Yᵢ, . . . , Y_{N} where i varies over the range 1 to N, each delivering a respective time-varying electrical signal s₁, s₂, . . . , sᵢ, . . . , s_{N}.

The method consists in taking the time-varying electrical signals sᵢ(t) delivered by each of the sensors and representative of the sums of the signals Sⱼ emitted by the noise sources Xⱼ, The signals sᵢ(t) received on the N sensors on the basis of the sum of the contributions of the various sources is written as follows:

$${s}_{i} ( t ) = {\sum }_{j = 1}^{M} {A}_{ij} {S}_{j} ( t - \frac{{r}_{ij}}{c} )$$

where i=1 to N, rᵢⱼ is the distance between the noise source Xⱼ and the sensor Yᵢ, and c is the speed of sound in the ambient medium.

The term Aᵢⱼ represents the attenuation due to propagation together with the sensitivity factor of the sensors and is expressed as follows:
Aᵢⱼ=BᵢC(rᵢⱼ)
where i=1 to N and j=1 to M, where Bᵢ is the sensitivity coefficient of sensor Yᵢ and where C(rᵢⱼ) is the attenuation coefficient due to propagation over a distance rᵢⱼ.

The sensors Yᵢ are associated with respective electronic units (not shown) for amplifying and lowpass filtering the signals they pick up. The sensors are preferably matched in modulus and phase so that their sensitivities are identical. Thus, Bᵢ=G for i=1 to N.

Advantageously, in order to facilitate implementing the antenna of sensors as defined above, the sensors Yᵢ are placed relatively close to one another. Consequently, for remote sources, the distance rᵢⱼ is of the order of the distance rⱼ, i.e. the distance between the center of gravity of the sensors and the source Xⱼ. Thus, attenuation becomes a function of the distance rⱼ only with C(rᵢⱼ)=C(rⱼ), with i=1 to N and j=1 to M.

It can be deduced therefrom that:
Aᵢⱼ=G·C(rⱼ)=a(rⱼ)
where i=1 to N and j=1 to M and:

$${s}_{i} ( t ) = {\sum }_{j = 1}^{M} a ( {r}_{j} ) {S}_{j} ( t - \frac{{r}_{ij}}{c} )$$

where i =1 to N.

Since the amplitudes of the sources Xⱼ are unknown, the following equation can be written as follows, integrating the term a(rⱼ) in Sⱼ:

$${s}_{i} ( t ) = {\sum }_{j = 1}^{M} {S}_{j} ( t - \frac{{r}_{ij}}{c} )$$

where i=1 to N.

Using Fourier transforms, the expression for the signals sᵢ(t) becomes:

$${\hat{s}}_{i} ( ω ) = {\sum }_{j = 1}^{M} {\hat{ S}}_{j} ( ω ) \cdot {ⅇ}^{- J ω \frac{{r}_{ij}}{c}} \qquad ( 1 )$$

where i=1 to N
where ŝ and Ŝ are the Fourier transforms of s and S respectively and where ω is angular frequency.

This first equation (1) relates the received signals to the distance rᵢⱼ, i.e. to the positions of the sources Xⱼ.

As can be seen in FIG. 2, other relationships can be expressed, associated with geometrical considerations enabling the distances rᵢⱼ to be related to the unit vector nⱼ, which determines the direction defined by the center of gravity of the sensors and the source generating the signal Sⱼ. The position of the sensors is defined by the vector Cᵢ constructed from the positions of the sensors Yᵢ and the position of their center of gravity. A development restricted to the first order of rᵢⱼ then provides:
rᵢⱼ≈rⱼ−<nⱼ, cᵢ> (2)
where i=1 to N and j=1 to M, and where <., .> is the scalar product.

Thus, by replacing rᵢⱼ by the approximate expression given in (2) and integrating the phase term:

$${ⅇ}^{- J ω \frac{{r}_{j}}{c}}$$

which depends only on the source Xⱼ in the magnitude Ŝⱼ(ω), equation (1) can be written:

$${\hat{s}}_{i} ( ω ) = {\sum }_{j = 1}^{M} {\hat{S}}_{j} ( ω ) \cdot {ⅇ}^{- J ω \frac{< {n}_{j} , {c}_{i} >}{c}} \qquad ( 3 )$$

where i=1 to N.

This relationship can also be expressed in matrix and vector form:

$${\hat{s}}_{i} ( ω ) = {\sum }_{j = 1}^{M} {\hat{S}}_{j} ( ω ) \cdot {T}_{j} ( ω ) \qquad ( 4 )$$

with, for ith coordinate of the vector Tⱼ:

$${( {T}_{j} )}_{i} = {ⅇ}^{- J ω \frac{< {n}_{j} , {c}_{i} >}{c}}$$

where i=1 to N. Or indeed:
s(ω)=T·S(ω) (5)
where T=matrix having the general term:

$${T}_{ij} = {ⅇ}^{- J ω \frac{< {n}_{j} , {c}_{i} >}{c}}$$

When the sources Xⱼ are not correlated, the signals Sⱼ can be determined from the signals sᵢ of the vectors nⱼ. Cross-correlation functions between Sᵢ and Sⱼ for i≠j are then minimized.

Once the minimization operation has been performed, after determining the directions nⱼ, it is also possible to discover the magnitudes Sⱼ.

If N=M, i.e. if there are as many sensors as sources, then the system (5) can in general be inverted.

If N≧M, the problem can be reduced to a square system by premultiplying by:
^{t}T*
i.e. by the conjugate transposed matrix of T· System (5) then becomes:
^{t}T*·s(ω)=^{t}T*·T·S(ω)
I.e.
S(ω)=(^{t}T*·T)⁻¹·^{t}T*·s(ω) (6)

With the signals Ŝ expressed formally in this way, the correlation coefficients Rᵢⱼ between the sources i and j are calculated formally by:

$${R}_{ij} = \frac{{\int }_{- ∞}^{+ ∞} {Γ}_{ij}^{2} ( τ ) ⅆ τ}{{Γ}_{ii} ( 0 ) \cdot {Γ}_{jj} ( 0 )} , i \neq j \qquad ( 7 )$$

where Γᵢⱼ can also be calculated formally from frequency magnitudes, giving:

$${R}_{ij} = \frac{{\int }_{- ∞}^{+ ∞} {\vert {\hat{S}}_{i} ( ω ) \vert}^{2} \cdot {\vert {\hat{S}}_{i} ( ω ) \vert}^{2} ⅆ ω}{{\int }_{- ∞}^{+ ∞} {\vert {\hat{S}}_{i} ( ω ) \vert}^{2} ⅆ ω \cdot {\int }_{- ∞}^{+ ∞} {\vert {\hat{S}}_{j} ( ω ) \vert}^{2} ⅆ ω} \qquad ( 8 )$$

The function for minimizing is then:

$$f ( {n}_{1} , … , {n}_{j} , … , {n}_{N} ) = {\sum }_{k \neq 1} {R}_{k1} \qquad ( 9 )$$

where the coefficients Rₖₗ are functions of the vectors nⱼ.

When the signals Sᵢ, Sⱼ are received with comparable amplitudes, the denominators of Rᵢⱼ are of the same order of magnitude and can then be replaced by 1 without spoiling the positions of the minimas. Calculating the Γᵢⱼ can then advantageously be performed in the time domain, when the range of variation in possible delays is small.

Once the directions defined by the vectors nⱼ have been determined, it is also possible to find the magnitudes Sⱼ from equation (6). Such a technique thus makes it possible to determine the natures of the sources Xⱼ.

The description below with reference to FIG. 3 gives an example of detecting and locating two noise sources X₁, X₂ that are not correlated (M=2), using two sensors Y₁, Y₂ (N=2).

In the frequency domain, the electrical signals s₁, s₂ delivered respectively by the sensors Y₁ and Y₂ and representative of the sum of the signals S₁, S₂ emitted by the noise sources X₁, X₂ are expressed as follows:

$$\{ \begin{matrix}{\hat{s}}_{1} ( ω ) = {ⅇ}^{- J {ωτ}_{11}} {\hat{S}}_{1} ( ω ) + {ⅇ}^{- J {ωτ}_{21}} {\hat{S}}_{2} ( ω ) \\ {\hat{s}}_{2} ( ω ) = {ⅇ}^{- J {ωτ}_{12}} {\hat{S}}_{1} ( ω ) + {ⅇ}^{- J {ωτ}_{22}} {\hat{S}}_{2} ( ω )\end{matrix}$$

where

$${τ}_{ij} = \frac{{r}_{ij}}{c}$$

is the propagation delay of the signal emitted by source i prior to reaching sensor j.

Inverting this system leads to:

$$\{ \begin{matrix}{\hat{S}}_{1} ( ω ) = \frac{{\hat{s}}_{1} ( ω ) {ⅇ}^{- J {ωτ}_{22}} - {\hat{s}}_{2} ( ω ) {ⅇ}^{- J {ωτ}_{21}}}{{ⅇ}^{- J ω ( {τ}_{11} + {τ}_{22} )} - {ⅇ}^{- J ω ( {τ}_{12} + {τ}_{21} )}} \\ {\hat{S}}_{2} ( ω ) = \frac{{\hat{s}}_{2} ( ω ) {ⅇ}^{- J {ωτ}_{11}} - {\hat{s}}_{1} ( ω ) {ⅇ}^{- J {ωτ}_{12}}}{{ⅇ}^{- J ω ( {τ}_{11} + {τ}_{22} )} - {ⅇ}^{- J ω ( {τ}_{12} + {τ}_{21} )}}\end{matrix}$$

The cross-correlation function between the source signals S₁ and S₂ is written:

$${Γ}_{12} ( τ ) = {\int }_{- ∞}^{+ ∞} {\hat{S}}_{1} ( ω ) \cdot {\hat{S}}_{2} ( ω ) {ⅇ}^{J ωτ} ⅆ ω$$

for the delay τ.

Replacing Ŝ₁(ω) and Ŝ₂(ω), it becomes:

$${Γ}_{12} ( τ ) = {\int }_{- ∞}^{+ ∞} \frac{N ( ω )}{{\vert D ( ω ) \vert}^{2}} ⅆ ω$$

whence

$$N ( ω ) = [ \begin{matrix}- {\vert {\hat{s}}_{1} ( ω ) \vert}^{2} {ⅇ}^{J ω ( {τ}_{12} - {τ}_{22} )} - {\vert {\hat{s}}_{2} ( ω ) \vert}^{2} {ⅇ}^{J ω ( {τ}_{11} - {τ}_{21} )} + \\ {\hat{s}}_{1} ( ω ) {\hat{s}}_{2}^{*} ( ω ) {ⅇ}^{J ω ( {τ}_{11} - {τ}_{22} )} + {\hat{s}}_{1}^{*} ( ω ) {\hat{s}}_{2} ( ω ) {ⅇ}^{J ω ( {τ}_{12} - {τ}_{21} )}\end{matrix} ] \cdot {ⅇ}^{J ωτ}$$

and

$${\vert D ( ω ) \vert}^{2} = 4 {sin}^{2} \frac{ω}{2} ( {τ}_{11} + {τ}_{22} - {τ}_{12} - {τ}_{21} )$$

A sample (but sub-optimal) solution in this case consists in optimizing the numerator N only.

The cross-correlation Γ₁₂ can then be approximated by:

$${Γ}_{12} ( τ ) = {\int }_{- ∞}^{+ ∞} N ( ω ) ⅆ ω$$

Replacing N(ω) by its value an expression is obtained which is a function only of the γᵢⱼ corresponding to the autocorrelations and cross-correlations between the measured signals sᵢ and sⱼ:
Γ₁₂(τ)≅−γ₁₁(τ+τ₁₂−τ₂₂)−γ₂₂(τ+τ₁₁−τ₂₁ )+γ₁₂(τ+τ₁₁−τ₂₂)+γ₂₁(τ+τ₁₂−τ₂₁)

It is recalled that the distance rᵢⱼ can be approximated by:
rᵢⱼ≈rⱼ−<nⱼ, cᵢ>

Thus, replacing rᵢⱼ in this approximate expression and integrating the phase term

$${ⅇ}^{- J ω \frac{{r}_{j}}{c}}$$

in Sⱼ(ω) finally leads to an expression of the estimator of Γ₁₂ which is as follows:

$$\begin{matrix}{Γ}_{12} ( τ ) \approx - {γ}_{11} ( τ - \frac{< {n}_{2} , {c}_{1} >}{c} + \frac{< {n}_{2} , {c}_{2} >}{c} ) - \\ {γ}_{22} ( τ - \frac{< {n}_{1} , {c}_{1} >}{c} + \frac{< {n}_{1} , {c}_{2} >}{c} ) + \\ {γ}_{12} ( τ - \frac{< {n}_{1} , {c}_{1} >}{c} + \frac{< {n}_{2} , {c}_{2} >}{c} ) + \\ {γ}_{21} ( τ - \frac{< {n}_{2} , {c}_{1} >}{c} + \frac{< {n}_{1} , {c}_{2} >}{c} )\end{matrix}$$

where nⱼ=the unit vector of (OXᵢ) with i=1, 2. However:

$$< {n}_{1} , {c}_{1} >= - \frac{D}{2} cos {θ}_{1} \\ < {n}_{1} , {c}_{2} >= \frac{D}{2} cos {θ}_{1} \\ < {n}_{2} , {c}_{1} >= - \frac{D}{2} cos {θ}_{2} \\ < {n}_{2} , {c}_{2} >= - \frac{D}{2} cos {θ}_{2}$$

Where the distance between sensors is written is D. Then:

$${Γ}_{12} ( τ ) \approx - {γ}_{11} ( τ + \frac{D}{c} cos {θ}_{1} ) \\ - {γ}_{22} ( τ + \frac{D}{2} cos {θ}_{1} ) + \\ {γ}_{12} ( τ + \frac{D}{2 c} ( cos {θ}_{1} + cos {θ}_{2} ) ) + \\ {γ}_{21} ( τ + \frac{D}{2 c} ( cos {θ}_{1} + cos {θ}_{2} ) )$$

The functional to be minimized relative to (θ₁, θ₂) is thus:

$${R}_{12} = {\int }_{- ∞}^{+ ∞} {Γ}_{12}^{2} ( τ ) ⅆ τ$$

Sign ambiguity between θ₁ and θ₂ is removed by analyzing the half-plane containing the sources and assumed to be known a priori.

The invention is not limited to the examples described and shown, since various modifications can be made thereto without going beyond this ambit.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever. Also, any preceding examples can be repeated with similar success by substituting the generically or specifically described reactants and/or operating conditions of this invention for those used in such examples.

Throughout the specification and claims, all temperatures are set forth uncorrected in degrees Celsius and, all parts and percentages are by weight, unless otherwise indicated.

The entire disclosure of all applications, patents and publications, cited herein are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

1. A method of detecting and locating noise sources each emitting respective signals Sⱼ where j=1 to M, detection being provided by means of acoustic wave or vibration sensors each delivering a respective time-varying electrical signal sᵢ with i varying from 1 to N, the method consisting:
in taking the time-varying electrical signals delivered by the sensors, each signal sᵢ(t) delivered by a sensor being the sum of the signals Sⱼ emitted by the noise sources;
in amplifying and filtering the taken time-varying electrical signals;
in digitizing the electrical signals;
in calculating the functional
$$f ( {n}_{1} , … , {n}_{j} , … , {n}_{N} ) = {\sum }_{k \neq 1}^{} {R}_{k1}$$
with the coefficients Rₖₗ being a function of the vectors nⱼ giving the directions of the noise sources; and
in minimizing the functional f in such a manner as to determine the directions nⱼ of the noise sources.

2. A method according to claim 1, wherein, in order to minimize the functional f, the method consists in:
calculating the Fourier transforms of the signals sᵢ(t) delivered by the sensors;
formally calculating the coefficients Rᵢⱼ:
$${R}_{ij} = \frac{{\int }_{- ∞}^{+ ∞} {\vert {\hat{S}}_{i} ( ω ) \vert}^{2} \cdot {\vert {\hat{S}}_{i} ( ω ) \vert}^{2} ⅆ ω}{{\int }_{- ∞}^{+ ∞} {\vert {\hat{S}}_{i} ( ω ) \vert}^{2} ⅆ ω \cdot {\int }_{- ∞}^{+ ∞} {\vert {\hat{S}}_{j} ( ω ) \vert}^{2} ⅆ ω}$$
and minimizing the functional f in order to determine the directions nⱼ of the selected noise sources.

3. A detection method according to claim 1, wherein, in order to minimize the functional f, the method consists:
in formally calculating the correlation coefficient Rᵢⱼ:
$${R}_{ij} = \frac{{\int }_{- ∞}^{+ ∞} {Γ}_{ij}^{2} ( τ ) ⅆ τ}{{Γ}_{ii} ( 0 ) \cdot {Γ}_{jj} ( 0 )}$$
where Γᵢⱼ is the cross-correlation function between the signals Sᵢ and Sⱼ.

4. A detection method according to claim 1, wherein, after performing the minimization operation, the method consists in calculating the source vector:
S(w)=(^{t}T*·T)⁻¹·^{t}T*·s(ω)
in order to find the characteristics of the noise sources.
